(19) United States
(12) Patent Application Publication
(10) Pub. No.: US 2015/0238579 A1
(43) Pub. Date: Aug. 27, 2015

(54) METHODS FOR ASSEMBLING A PRODUCT FOR MODULATING AN IMMUNOMODULATORY RESPONSE IN HUMANS

(71) Applicant: INVESTFOOD, LLC, Granada (ES)

(72) Inventors: Alberto Baños Arjona, Granada (ES); Julio J. Gálvez Peralta, Granada (ES); Enrique Guillamón Ayala, Granada (ES); Cristina Núñez Lechado, Granada (ES); Francisco Maroto Caba, Granada (ES); Maria Elena Rodriquez Cabezas, Granada (ES)

(73) Assignee: INVESTFOOD, LLC, Granada (ES)

(21) Appl. No.: 14/191,158

(22) Filed: Feb. 26, 2014

(51) Int. Cl. A61K 39/00; A23L 2/52; A23L 1/30

(52) U.S. Cl. CPC A61K 39/00; A23L 1/30; A23L 2/52; A61K 2039/58; A23V 2002/00

(57) ABSTRACT

Embodiments of the invention provide a method for assembling a propyl propane thiosulfonate (PTSO) product for modulating immune function in healthy and sick humans. The method includes selecting a PTSO based compound for incorporation into a PTSO product. The PTSO based compound includes PTSO having a formula R—SOa-S—R, where R represents n-propyl group (—CH₂—CH₂—CH₃) and a is 2. The PTSO based compound is then incorporated into a PTSO product.

### BACKGROUND OF THE INVENTION

1. Field of the Invention

The present invention relates to immunology and, more particularly, to the modulation of immune response in humans.

2. Description of the Related Art

Immunology is the biomedical science that covers the study of all aspects of the immune system in organisms. The normal functioning of the immune system is crucial to the health of human beings. Immunomodulators, such as tolerogens, azathioprine, 6-mercaptopurine, methotrexate, cyclosporine, levamisole, thalidomide, and tacrolimus, have been the focus of immunology research as immunomodulators have been found to enhance immune system responsiveness, and thereby improve health and reduce disease risks, through immunocompetence modulation.

An immunomodulator is any substance that helps to regulate the immune system to optimize immune response. Immunomodulators may act as immunosuppressants by inhibiting the immune response or as immunostimulants by stimulating the immune response. Immunosuppressants are helpful in treating inflammatory and autoimmune diseases, while immunostimulants can improve the immune function of people with chronic infectious diseases, immunodeficiency disorders, and cancer.

Immunomodulators can act at two levels of the immune system, either by modulating the specific immune response by enhancing the synthesis of immunoglobulins (immunological adjuvant) or modulating the specific immune responses through the regulation of the production of soluble mediators. In the first case, the immunomodulator exerts an immunoreaction towards a particular antigen, as is the case of immunological adjuvants. Immunological adjuvants are chemical or biological preparations which make the immune response more effective, making it faster, strong and durable. They play a very important role in the processes of induction and activation of the immune response by inducing the recruitment and activation of accessory cells and other co-stimulatory. In the second case, the stimulation or suppression of the immune response is produced without the need for activation by a given antigen, such as the regulation of cytokine synthesis.

Cytokines are a diverse group of proteins, peptides or glycoproteins that act as hormonal regulators in the organism such as interleukins and interferons. Cytokines are regulators of host responses to infection, immune responses, and inflammation. Some cytokines are anti-inflammatory and serve to reduce inflammation and promote healing of the injury produced, while others are pro-inflammatory, recruiting granulocytes and lymphocytes. However, excessive inflammation is sometimes the pathogenicity of certain diseases. However, there are a lack of chemicals on the market capable of acting at both levels, hence the need to select more potent, effective and safe immunomodulators, which are able to develop an effective immune response with potential application in clinical practice and functional nutrition.

### BRIEF SUMMARY OF THE INVENTION

Embodiments of the present invention relate to a functional therapeutic composition and provide novel and non-obvious methods for assembling a product for modulating immune function in humans. In an embodiment of the invention, a PTSO based compound can be selected for incorporation into a PTSO product. The PTSO based compound includes PTSO having a formula R—SOa-S—R, where R represents n-propyl group (—CH₂—CH₂—CH₃) and a is 2. Upon selecting the PTSO based compound, the PTSO based compound can be incorporated into the PTSO product. A dose of the PTSO product can include an amount of the PTSO based compound between at least zero point zero one milligrams (0.01 mg) and no more than two thousand milligrams (2000 mg).

In a different embodiment of the invention, an amount of PTSO can be selected for a PTSO based compound. Upon creating the PTSO based compound containing the selected amount of PTSO, the PTSO based compound can be incorporated into a PTSO product. The amount of PTSO selected is such so a dose of the PTSO product contains an amount of the PTSO based compound between at least zero point zero one milligrams (0.01 mg) and no more than two thousand milligrams (2000 mg).

In a further embodiment of the invention, propyl propane thiosulfonate (PTSO) having the formula R—SOa-S—R, where R represents n-propyl group (—CH₂—CH₂—CH₃) and a is 2, can be administered to humans via a PTSO product in order to modulate immune functions.

In yet further embodiments, a PTSO product (and, thus, a PTSO based compound) can be administered to humans to treat and/or to prevent chronic infectious diseases, immunodeficiency disorders, cancer, multiple inflammatory bowel diseases, and autoimmune and chronic inflammatory diseases and conditions.

Additional aspects of the invention will be set forth in part in the description which follows, and in part will be obvious from the description, or may be learned by practice of the invention. The aspects of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE SEVERAL VIEWS OF THE DRAWINGS

The accompanying drawings, which are incorporated in and constitute part of this specification, illustrate embodiments of the invention and together with the description, serve to explain the principles of the invention. The embodiments illustrated herein are presently preferred; it being understood, however, that the invention is not limited to the precise arrangements and instrumentalities shown, wherein:

FIG. 1 is a pictorial illustration of a method for modulating immune function in humans;

FIG. 2A is a flow chart illustrating a method for modulating immune function in humans;

FIG. 2B is a flow chart illustrating the assembly of a PTSO product for modulating immune function in humans in an embodiment of the invention;

FIG. 2C is a flow chart illustrating the assembly of a PTSO product for modulating immune function in humans in a different embodiment of the invention;

FIG. 3 is a bar chart illustrating the performance of PTSO in reducing the production of IL-1β in a dose-dependent manner;

FIG. 4 illustrates that PTSO modulates the production of IL-6;

FIG. 5 illustrates PTSO immunomodulatory activity in the production of IL-8 cytokine on human epithelial colorectal adenocarcinoma cells (Caco-2); and,

FIG. 6 illustrates the modulating immune function in the production of interferon gamma (IFN-γ) in human leukemic T lymphocytes.

### DETAILED DESCRIPTION OF THE INVENTION

Embodiments of the invention provide for methods for assembling a product for modulating immune function. Specifically, in an embodiment of the invention, a propyl propane thiosulfonate (PTSO) product can be assembled by selecting an amount of PTSO for a PTSO based compound to be incorporated into a PTSO product. The PTSO based compound can include PTSO having the formula R—SOa-S—R, where R represents n-propyl group (—CH₂—CH₂—CH₃) and a is 2. The PTSO based compound can then be incorporated into the PTSO product upon creating the PTSO based compound containing the selected amount of PTSO. The amount of the PTSO selected is the amount necessary so to have a dose of the PTSO product (each application) contains an amount of the PTSO based compound between at least zero point zero one milligrams (0.01 mg) and no more than two thousand milligrams (2000 mg). The PTSO product can then be administered to a human to modulate immune function. In this way, the PTSO based compound via a PTSO product can be used to improve an immune response in a person by producing an immunomodulatory effect in different pathological processes, thereby, preventing and treating chronic infectious diseases, immunodeficiency disorders, cancer, and systemic and bowel inflammatory diseases.

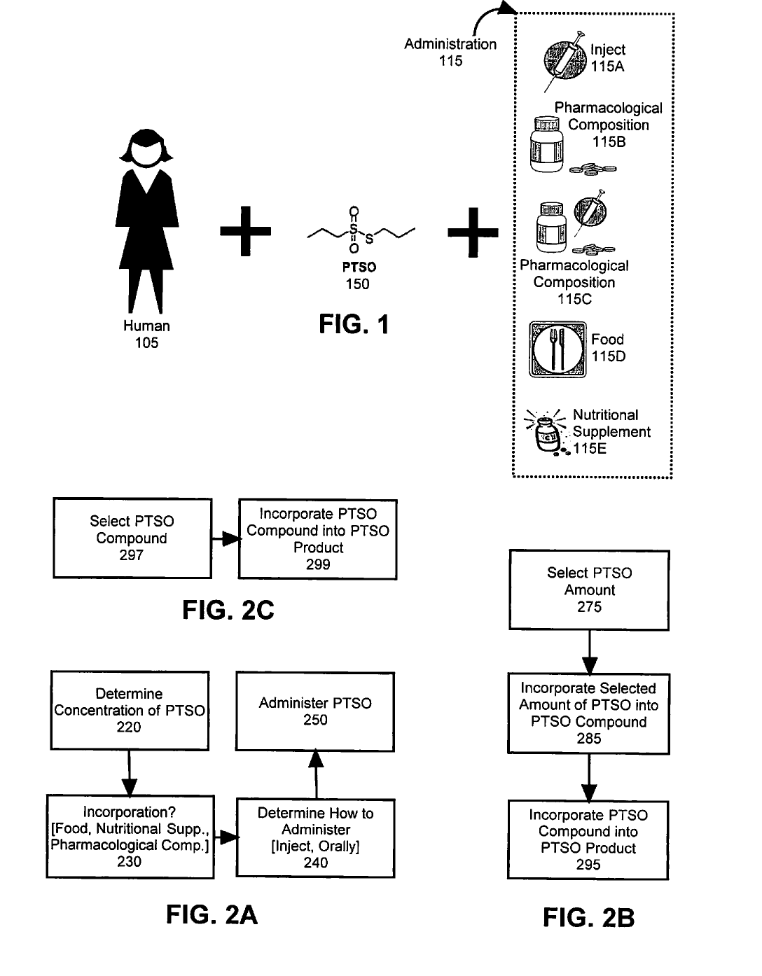

In further illustration, FIG. 1 pictorially shows a process for modulating immune function. As shown in FIG. 1, a compound including PTSO 150 can be administered to a human 105—healthy or sick. The administration 115 of the PTSO 150 (and thus the PTSO based compound via a PTSO product) can be accomplished via an injection 115A, incorporation with a pharmacological composition, via orally administration 115B, via incorporation within a pharmacological composition and thusly injected 115C, via incorporation within a food product and/or a food supplement and thusly orally administered 115D, or via incorporation into a nutritional supplement 115E, such as a vitamin. Subsequent to administration of PTSO 150 in the PTSO based compound, immune function can be modulated in humans. In addition, after the continued administration of PTSO, the immune response of the human is improved by immunomodulatory mechanisms triggered.

In yet further illustration of the invention, FIG. 2A is a flow chart illustrating a process for modulating immune function in humans. Beginning in block 220, the desired concentration of PTSO in a PTSO based compound to be administered can be determined based upon the desired dose. A dose of the PTSO product can contain an amount of the PTSO based compound between at least zero point zero one milligrams (0.01 mg) and no more than two thousand milligrams (2000 mg). In a different embodiment, a dose can be between zero point one and one thousand milligrams (0.1-1000 mg). In yet a different embodiment, a dose can be between zero point five and five hundred fifty milligrams (0.5-550 mg).

As illustrated in block 230, a determination can be made as to whether to incorporate the PTSO based compound (and thus PTSO itself), into food and/or a food supplement, nutritional supplements, and/or pharmacological compounds resulting in a PTSO product. Of note, the PTSO based compound can be administered by itself (just PTSO, with no other additives). A determination can then be made as to how to administer the PTSO based compound (and thus, a PTSO product), as illustrated in block 240. For example, the PTSO based compound can be injected or orally administered. Thereafter, the PTSO based compound can be administered, as shown in block 250, so as to modulate immune function in humans as well as to treat and/or to prevent chronic infectious diseases, immunodeficiency disorders, cancer, multiple inflammatory bowel diseases, and autoimmune and chronic inflammatory diseases and conditions.

PTSO corresponds to the following formula:

R—SOa-S—R

in which R represents n-propyl group (—CH₂—CH₂—CH₃), and a is 2 for PTSO. Of note, PTSO is also known as dipropyl thiosulfonate, propyl propane thiosulfonate or 1-propylsulfonylsulfanylpropane (CAS Number 1113-13-9).

Of note, in addition to modulating immune function, which can include modulating cytokine production as well as other markers in humans, it is also contemplated that the invention can also be used to prevent or treat chronic infectious diseases and immunodeficiency disorders, such as acquired immune deficiency syndrome (AIDS), cancer, multiple inflammatory bowel diseases and other autoimmune and chronic inflammatory diseases. Inflammatory diseases can include, but are not limited to, inflammatory bowel diseases (e.g. ulcerative colitis and Crohn's disease) ileitis, irritable bowel syndrome, celiac disease, and chronic inflammatory intestinal disease. It is further contemplated that the present invention can be used as an immunostimulator agent. Specifically, propyl propane thiosulfonate (PTSO) can be used to potentiate the immune response to an antigen and/or modulate it towards the desired immune response (e.g. to improve pre-existing immunity, to enhance synthesis of specific immunoglobulins post-antigen exposure, and to enhance antigen-specific immune responses when used in combination with specific vaccine antigens or as an immunologic adjuvant).

Of further note, PTSO can be used alone or in combination with other immunomodulatory products and/or pharmacological carriers. Depending on the application dose, PTSO can act as an immunostimulant or an immunosuppressive compound with respect to immunoglobulins and cytokine production. For example at 0.1 mM, a PTSO based compound can increase the production of IL-6 in THP-1 cells, whereas in contrast, starting from more than 1 mM dose a clear immunosuppressive effect is observed for this interleukin. However, the dose-effect relationship will depend specifically on the type of cytokine and specific cell type.

Therefore, PTSO can modulate immune response in the presence of disease, but can also enhance an immune response in a healthy person. As indicated herein, PTSO can be administered to healthy or sick individuals orally, including directly, by being incorporated into food and/or food supplements, nutritional supplements, or in pharmacological compositions (including drugs and other immunomodulatory products). Formulations of the invention suitable for oral administration can be in the form of capsules, pills, lozenges, powders, granules, or as a solution, a suspension in an aqueous or non-aqueous liquid, an emulsion, a beverage, or as syrup. In addition, PTSO can be administered to the patient directly by injection alone or as part of a pharmacological composition.

In yet further illustration of the invention, FIG. 2B shows a flow chart illustrating the assembly of a PTSO product for modulating immune function in humans. In block 255, an amount of PTSO can be selected. The amount of PTSO selected is the amount necessary (the effective amount) to create a PTSO based compound that results in a dose of the PTSO product being between at least zero point zero one milligrams (0.01 mg) and no more than two thousand milligrams (2000 mg). In other words, a dose of the PTSO product contains an amount of the PTSO based compound between at least zero point zero one milligrams (0.01 mg) and no more than two thousand milligrams (2000 mg).

The selected amount of PTSO can be incorporated into a PTSO based compound so to create the PTSO based compound with the desired concentration, as shown in block 285. As illustrated in block 295, the PTSO based compound can then be incorporated into a PTSO product. In the PTSO product can be in any suitable formulation for oral administration. For instance, the PTSO product can be in the form of a capsule, a pill, a lozenge, a powder, and/or a granule. The PTSO product can also be in the form of a solution, a suspension in an aqueous or non-aqueous liquid, an emulsion, a beverage, and/or a syrup. Further, the PTSO product can be a pre-filled syringe, in which the pre-filled syringe is filed with a PTSO solution. The PTSO solution can comprise a PTSO based compound having a selected concentration which can be used to create a PTSO product in which a dose of the PTSO product contains an amount of PTSO based compound between at least zero point zero one milligrams (0.01 mg) and no more than two thousand milligrams (2000 mg). As the PTSO product is to be administered to a human, the molecule is food grade.

In even yet a further illustration of the invention, FIG. 2C shows a flow chart illustrating the assembly of a PTSO product for modulating immune function in humans. As shown in block 297, a PTSO based compound can be selected. More specifically, the PTSO based compound can be selected from a set of “pre-made” PTSO based compounds. The PTSO based compound can then be incorporated into a PTSO product, as illustrated in block 299. Depending on the selected PTSO based compound, the PTSO product can include an amount of the PTSO based compound between at least zero point zero one milligrams (0.01 mg) and no more than two thousand milligrams (2000 mg) for each dose of the PTSO product. As indicated herein, the PTSO product can be in the form of a capsule, a pill, a lozenge, a powder, and/or a granule. The PTSO product can also be in the form of a solution, a suspension in an aqueous or non-aqueous liquid, an emulsion, a beverage, and/or a syrup. Further, the PTSO product can be a pre-filled syringe, in which the pre-filled syringe is filed with a PTSO solution. The PTSO solution can comprise a PTSO based compound having a desired concentration. As the PTSO product is to be administered to a human and/or consumed by a human, the molecule is food grade.

Of note, as understood herein, PTSO can be the drug itself which can be dissolved in an aqueous or non-aqueous solution and/or combined with dry ingredients to form a PTSO based compound. The PTSO based compound can then be incorporated with immunomodulatory products, pharmacological carriers as well as binders, etc. to form a PTSO product (pill, drink, etc.). It should be understood that immunomodulatory products, pharmacological carriers, etc. can be added as part of creating the PTSO based compound as well. Additionally, it is understood that a dose is a specific quantity of a therapeutic drug or agent taken at any one time.

In further illustration of the invention, the following examples are presented. Of note, as will be understood by one skilled in the art, the invention is not limited to just these examples.

### Example 1

In this example, an evaluation of immunosuppressive activity of PTSO on human acute monocytic leukemia cell line (THP-1) was studied.

The effects of different concentrations of PTSO on cytokine production (IL-1β and IL-6) in the human acute monocytic leukemia cell line THP-1 activated by lipopolysaccharide (LPS) and phorbol 12-myristate 13-acetate (PMA) were evaluated. Of note, IL-1β and IL-6 cytokines have been reported to play a key role in the activated immune response that takes place in different inflammatory and/or immune conditions.

The human acute monocytic leukemia cell line THP-1 was obtained from the Cell Culture Unit of the University of Granada (Granada, Spain) (ECACC reference number: 88081201). These cells were cultured in Roswell Park Memorial Institute (RPMI) 1640 medium (Sigma, St Louis, Mo., USA) supplemented with 10% fetal bovine serum (Sigma), 2 mM glutamine and 0.05 mM mercaptoethanol in a humidified 5% carbon dioxide (CO₂) atmosphere at 37° C. The cultured cells were seeded onto 24-well plates at a density of 5×10⁵ cells/ml and pre-incubated for twenty four (24) hours with the different concentrations of the test compound (ranging from 0.1 to 25 μM of PTSO), and then stimulated with 1 μg/ml LPS and 1 μM phorbol 12-myristate 13-acetate (PMA). After twenty-four hours, the supernatants were collected and centrifuged at 1,000 g for 10 min, and then stored at −80° C. for the determination of IL-1β and IL-6 levels by an enzyme-linked immunosorbent assay (ELISA) (GE Healthcare, Little Chalfont, UK).

### Example 2

In Example 2, an evaluation of immunomodulatory activity of PTSO on human epithelial colorectal adenocarcinoma cells was conducted.

The effects of different concentrations of PTSO on cytokine production (IL-8) in the epithelial cell line human epithelial colorectal adenocarcinoma cells (CACO-2) activated by IL-1β were evaluated. IL-8 acts as a neutrophil attractant, which participates in the neutrophil infiltration that occurs in mucosal sites affected by an inflammatory process.

The human colonic adenocarcinoma cell line CACO-2 was obtained from the Cell Culture Unit of the University of Granada (Granada, Spain) (ECACC reference number: 86010202). Cells from the human colonic adenocarcinoma cell line CACO-2 were cultured in RPMI 1640 (Sigma) supplemented with 10% fetal bovine serum (Sigma), 2 mM glutamine and 0.05 mM mercaptoethanol in a humidified 5% CO₂ atmosphere at 37° C. The cultured cells were seeded onto 24-well plates until confluence and pre-incubated for twenty-four hours (24 hr) with the different concentrations of the test compound (ranging from 0.1 to 25 μM of PTSO), and then stimulated with 1 ng/ml IL-1β. After twenty four hours (24 hr), the supernatants were collected and centrifuged at 1,000 g for 10 min, and stored at −80° C. for the determination of IL-8 levels by ELISA (GE Healthcare). The effects of the different concentrations of PTSO on cell viability were assessed by using the methylthiazol terazolium (MTT) assay (Promega), following the same protocol of incubation of the test compound similar to that described above.

### Example 3

The objective of Example 3 was to study the immunomodulated effect of propyl propane thiosulphonate (PTSO) on human leukemic T lymphocytes.

The human T cell line JURKAT E6.1 was obtained from the Cell Culture Unit of the University of Granada (Granada, Spain) (ECACC reference number: 88042803). Cells from the human T cell line JURKAT E6.1 were cultured in RPMI 1640 (Sigma) supplemented with 10% fetal bovine serum (Sigma), 2 mM glutamine and 0.05 mM mercaptoethanol in a humidified 5% CO₂ atmosphere at 37° C. The cultured cells from the human T cell line JURKAT E6.1 were seeded onto 24-well plates at the concentration of 500,000 cells/ml and pre-incubated for 2 and 24 hours with the different concentrations of the test compound (ranging from 0.1 to 25 μM of PTSO), and then stimulated with 40 nM ionomycin and 0.5 μM of PMA. After twenty four hours (24 hours), the supernatants were collected and centrifuged at 1,000 g for 10 min, and stored at −80° C. for the determination of IFN-γ levels by ELISA (GE Healthcare).

Exemplary Results

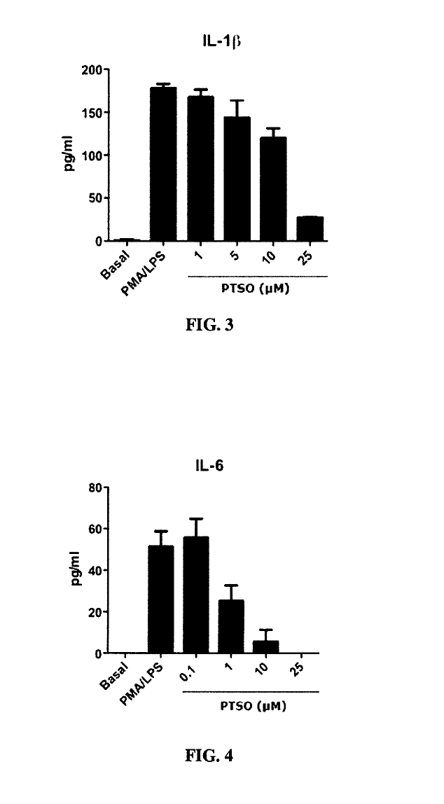

The results revealed that PTSO was able to reduce the production of IL-1β in a dose-dependent manner, as illustrated in FIG. 3.

In addition, FIG. 4 illustrates that PTSO can modulate the production of IL-6.

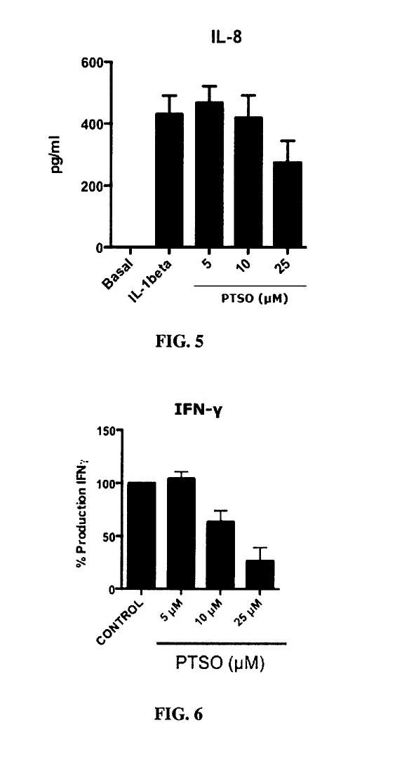

It is further noted that PTSO demonstrated immunomodulatory activity on the production of IL-8 cytokine on human epithelial colorectal adenocarcinoma cells (Caco-2), as shown in FIG. 5.

Furthermore, FIG. 6 illustrates the modulating immune function on the production of interferon gamma (IFN-γ) in human leukemic T lymphocytes.

The viability assays revealed that concentrations of PTSO up to 25 μM did not significantly affect the viability of THP-1, CACO-2, and Jurkat cells. Therefore, different effects on cytokine production could not be attributed to the action of compound on cell viability. As a consequence, these effects show an immunomodulatory response of the compound independent of any cytotoxic effect.

Having thus described the invention of the present application in detail and by reference to embodiments thereof, it will be apparent that modifications and variations are possible without departing from the scope of the invention defined in the appended claims as follows:

We claim:

1. A method for assembling a propyl propane thiosulfonate (PTSO) product for modulating immune function in humans comprising:
selecting an amount of PTSO for a PTSO based compound for incorporation into a PTSO product, the PTSO based compound comprising PTSO having a formula R—SOa-S—R, where R represents n-propyl group (—CH₂—CH₂—CH₃) and a is 2; and,
incorporating the PTSO based compound into the PTSO product upon creating the PTSO based compound containing the selected amount of PTSO, a dose of the PTSO product containing an amount of the PTSO based compound between at least zero point zero one milligrams (0.01 mg) and no more than two thousand milligrams (2000 mg).

2. The PTSO product of claim 1, wherein the PTSO product is a pill.

3. The PTSO product of claim 1, wherein the PTSO product is a capsule.

4. The PTSO product of claim 1, wherein the PTSO product is a beverage.

5. The PTSO product of claim 1, wherein the PTSO product is a food supplement.

6. The PTSO product of claim 1, wherein the PTSO product is a nutritional supplement.

7. The PTSO product of claim 1, wherein the PTSO product is a pharmacological composition.

8. The PTSO product of claim 1, wherein the PTSO based compound is a PTSO solution.

9. The PTSO product of claim 8, further comprising incorporating the PTSO solution into a syringe to create a pre-filled syringe, the pre-filled syringe being the PTSO product.

10. The PTSO product of claim 1, further comprising incorporating one or more immunomodulatory products with the PTSO product.

11. The PTSO product of claim 1, further comprising incorporating one or more immunomodulatory products with the PTSO based compound.

12. A method for assembling a propyl propane thiosulfonate (PTSO) product for modulating immune function in humans comprising:
selecting a PTSO based compound for incorporation into a PTSO product, the PTSO based compound comprising PTSO having a formula R—SOa-S—R, where R represents n-propyl group (—CH₂—CH₂—CH₃) and a is 2; and,
incorporating the PTSO based compound into the PTSO product upon selecting the PTSO based compound.

13. The PTSO product of claim 12, wherein the PTSO product is a food supplement.

14. The PTSO product of claim 12, wherein the PTSO product is a nutritional supplement.

15. The PTSO product of claim 12, wherein the PTSO product is a pharmacological composition.

16. The PTSO product of claim 12, wherein the PTSO based compound is a PTSO solution.

17. The PTSO product of claim 16, further comprising incorporating the PTSO solution into a syringe to create a pre-filled syringe, the pre-filled syringe being the PTSO product.

18. The PTSO product of claim 12, further comprising incorporating one or more immunomodulatory products with the PTSO product.

19. The PTSO product of claim 12, further comprising incorporating one or more immunomodulatory products with the PTSO based compound.

20. The PTSO product of claim 12, further comprising a dose of the PTSO product containing an amount of the PTSO based compound between at least zero point zero one milligrams (0.01 mg) and no more than two thousand milligrams (2000 mg).
